(19) United States
(12) Patent Application Publication
(10) Pub. No.: US 2005/0244503 A1
(43) Pub. Date: Nov. 3, 2005

(54) Small-particle pharmaceutical formulations of antiseizure and antidementia agents and immunosuppressive agents

(76) Inventors: Barrett E. Rabinow, Skokie, IL (US); Jane Werling, Arlington Heights, IL (US); Jamie T. Konkel, Island Lake, IL (US); Mark Doty, Grayslake, IL (US); Christine L. Rebbeck, Algonquin, IL (US)

Correspondence Address: Michael C. Mayo;Baxter International Inc., DF3-2E, One Baxter Parkway, Deerfield, IL, 60015, US

(21) Appl. No.: 10/848,765

(22) Filed: May 19, 2004

Related U.S. Application Data
(60) Provisional application No. 60/471,581, filed on May 19, 2003.

(51) Int. Cl. A61K 9/14

(52) U.S. Cl. 424/489

(57) ABSTRACT

This invention pertains to the formulation of small-particle suspensions of anticonvulsants and antidementia, particularly carbamazepine, for pharmaceutical use. This invention also pertains to the formulation of small-particle suspensions of immunosuppressive agents, particularly cyclosporin, for pharmaceutical use.

### CROSS-REFERENCE TO RELATED APPLICATION

Not Applicable

### FEDERALLY SPONSORED RESEARCH OR DEVELOPMENT

Not Applicable.

### BACKGROUND OF THE INVENTION

1. Technical Field

This invention pertains to the formulation of small-particle suspensions of anticonvulsants, particularly carbamazepine, for pharmaceutical use. The advantages of these formulations include potentially higher drug loading with the possibility of minimizing side effects such as drowsiness, fatigue, dizziness, nystagmus or nausea. This invention also pertains to the formulation of small-particle suspensions of immunosuppressive agents, particularly cyclosporin, for pharmaceutical use.

2. Background Art

There is an ever increasing number of organic compounds being formulated for therapeutic or diagnostic effects that are poorly soluble or insoluble in aqueous solutions. Such drugs provide challenges to delivering them by the administrative routes detailed above. Compounds that are insoluble in water can have significant benefits when formulated as a stable suspension of sub-micron particles. Accurate control of particle size is essential for safe and efficacious use of these formulations. Particles must be less than seven microns in diameter to safely pass through capillaries without causing emboli (Allen et al., 1987; Davis and Taube, 1978; Schroeder et al., 1978; Yokel et al., 1981). One solution to this problem is the production of small particles of the insoluble drug candidate and the creation of a microparticulate or nanoparticulate suspension. In this way, drugs that were previously unable to be formulated in an aqueous based system can be made suitable for intravenous administration. Suitability for intravenous administration includes small particle size (<7 μm), low toxicity (as from toxic formulation components or residual solvents), and bioavailability of the drug particles after administration.

Preparations of small particles of water insoluble drugs may also be suitable for oral, pulmonary, topical, ophthalmic, nasal, buccal, rectal, vaginal, transdermal administration, or other routes of administration. The small size of the particles improves the dissolution rate of the drug, and hence improving its bioavailability and potentially its toxicity profiles. When administered by these routes, it may be desirable to have particle size in the range of 5 to 100 μm, depending on the route of administration, formulation, solubility, and bioavailability of the drug. For example, for oral administration, it is desirable to have particle size of less than about 7 μm. For pulmonary administration, the particles are preferably less than about 10 μm in size.

This invention pertains to the formulation of small-particle suspensions of anticonvulsants for pharmaceutical use. The advantages of these formulations include potentially higher drug loading with the possibility of minimizing side effects such as drowsiness, fatigue, dizziness, nystagmus or nausea. In particular, this invention entails formulations of tricyclic anticonvulsants having the general structure shown in FIG. 3.

This invention also pertains to the formulation of small-particle suspensions of cyclosporin for pharmaceutical use.

### SUMMARY OF THE INVENTION

The present invention provides a composition of an anticonvulsant or an immunosuppressive agent. The composition includes solid particles of the agent coated with one or more surface modifiers. The surface modifiers can be selected from anionic surfactants, cationic surfactants, zwitterionic surfactants, nonionic surfactants and surface active biological modifiers. The particles have an average effective particle size of from about 10 nm to about 100 microns. In a preferred embodiment, the anticonvulsant agent is a tricyclic anticonvulsant agent. In a more preferred embodiment, the tricyclic anticonvulsant agent is carbamazepine. In another preferred embodiment, the immunosuppressive agent is cyclosporin.

These and other aspects and attributes of the present invention will be discussed with reference to the following drawings and accompanying specification.

### BRIEF DESCRIPTION OF THE DRAWINGS

FIG. 1 shows a diagrammatic representation of one method of the present invention;

FIG. 2 show a diagrammatic representation of another method of the present invention; and

FIG. 3 shows the general structures of tricyclic anticonvulsant drugs.

### DETAILED DESCRIPTION OF THE INVENTION

The present invention is susceptible of embodiments in many different forms. Preferred embodiments of the invention are disclosed with the understanding that the present disclosure is to be considered as exemplifications of the principles of the invention and are not intended to limit the broad aspects of the invention to the embodiments illustrated. The present invention provides compositions and methods for forming small particles of an organic compound. An organic compound for use in the process of this invention is any organic chemical entity whose solubility decreases from one solvent to another. This organic compound might be a pharmaceutically active compound, which can be selected from therapeutic agents, diagnostic agents, cosmetics, nutritional supplements, and pesticides. In particular, the present invention provides compositions and methods for forming small particles of anticonvulsant and antidementia agents and immunosuppressive agents.

As used herein, “anticonvulsant agent” refers to agents that prevent, reduce, or stop convulsions or seizures. A seizure is an abnormal electrical discharge from the brain. It may affect a small focal area of the brain, or the entire brain (generalized). The area affected by the seizure loses its regular ability of function and may affect motor or sensory sites that the disabled part of the brain controls. For example, if an area of the brain that controls an arm has a seizure, the arm may shake repetitively. If a seizure affects the entire brain, all the extremities may shake uncontrollably. Some seizures may present with staring and unresponsiveness. Theoretically, any function of the brain—motor, smell, vision, or emotion may be individually affected by a seizure.

As used herein, “antidimentia agent” refers to agents prevent, reduce, or stop the course of development of dementia. Dementia is a clinical state characterized by loss of function in multiple cognitive domains. The most commonly used criteria for diagnoses is the DSM-IV (Diagnostic and Statistical Manual for Mental Disorders, American Psychiatric Association). Diagnostic features include memory impairment and at least one of the following: aphasia, apraxia, agnosia, and disturbances in executive functioning. Cognitive impairments must be severe enough to cause deficits in social and occupational functioning. Importantly, the decline must represent a decline from a previously higher level of functioning. There are approximately 70 to 80 different types of dementia. Some of the major disorders causing dementia are degenerative diseases (e.g., Alzheimer's, Pick's Disease), vascular dementia (e.g., multi-infarct dementia), anoxic dementia (e.g., cardiac arrest), traumatic dementia (e.g., dementia pugilistica [boxer's dementia]), infectious dementia (e.g., Creutzfeldt-Jakob Disease), toxic dementia (e.g., alcoholic dementia).

As used herein, “immunosuppressive agent” refers to agents that suppress the body's ability to elicit an immunological response to the presence of an antigen/allergen. For example, the ability to fight off disease or reject a transplanted organ. Another term for these agents is anti-rejection agents. Not only are they are used to treat organ rejection after transplantation, but many other diseases of immunological etiology such as Crohn's disease, rheumatoid arthritis, lupus, multiple sclerosis, and psoriasis.

The compositions of the present invention comprise the foregoing agents and, optionally, one or more additional therapeutic agents.

The therapeutic agents can be selected from a variety of known pharmaceuticals such as, but are not limited to: analgesics, anti-inflammatory agents, antihelmintics, anti-arrhythmic agents, antibiotics, anticoagulants, antidepressants, antidiabetic agents, antiepileptics, antifungals, antihistamines, antihypertensive agents, antimuscarinic agents, antimycobacterial agents, antineoplastic agents, antiprotozoal agents, immunosuppressants, immunostimulants, antithyroid agents, antiviral agents, anxiolytic sedatives, astringents, beta-adrenoceptor blocking agents, contrast media, corticosteroids, cough suppressants, diagnostic agents, diagnostic imaging agents, diuretics, dopaminergics, haemostatics, immuniological agents, lipid regulating agents, muscle relaxants, parasympathomimetics, parathyroid calcitonin, prostaglandins, radio-pharmaceuticals, sex hormones, anti-allergic agents, stimulants, sympathomimetics, thyroid agents, vasodilators, vaccines and xanthine. Antineoplastic, or anticancer agents, include but are not limited to paclitaxel and derivative compounds, and other antineoplastics selected from the group consisting of alkaloids, antimetabolites, alkylating agents and antibiotics.

Diagnostic agents include the x-ray imaging agent and contrast media. Examples of x-ray imaging agents include WIN-8883 (ethyl 3,5-diacetamido-2,4,6-triiodobenzoate) also known as the ethyl ester of diatrazoic acid (EEDA), WIN 67722, i.e., (6-ethoxy-6-oxohexyl-3,5-bis(acetamido)-2,4,6-triiodobenzoate; ethyl-2-(3,5-bis(acetamido)-2,4,6-triiodobenzoyloxy)butyrate (WIN 16318); ethyl diatrizoxyacetate (WIN 12901); ethyl 2-(3,5-bis(acetamido)-2,4,6-triiodobenzoyloxy)propionate (WIN 16923); N-ethyl 2-(3,5-bis(acetamido)-2,4,6-triiodobenzoyloxy acetamide (WIN 65312); isopropyl 2-(3,5-bis(acetamido)-2,4,6-triiodobenzoyloxy) acetamide (WIN 12855); diethyl 2-(3,5-bis(acetamido)-2,4,6-triiodobenzoyloxy malonate (WIN 67721); ethyl 2-(3,5-bis(acetamido)-2,4,6-triiodobenzoyloxy) phenylacetate (WIN 67585); propanedioic acid, [[3,5-bis(acetylamino)-2,4,5-triodobenzoyl]oxy]bis(1-methyl)ester (WIN 68165); and benzoic acid, 3,5-bis(acetylamino)-2,4,6-triodo4-(ethyl-3-ethoxy-2-butenoate) ester (WIN 68209). Preferred contrast agents include those which are expected to disintegrate relatively rapidly under physiological conditions, thus minimizing any particle associated inflammatory response. Disintegration may result from enzymatic hydrolysis, solubilization of carboxylic acids at physiological pH, or other mechanisms. Thus, poorly soluble iodinated carboxylic acids such as iodipamide, diatrizoic acid, and metrizoic acid, along with hydrolytically labile iodinated species such as WIN 67721, WIN 12901, WIN 68165, and WIN 68209 or others may be preferred.

A description of these classes of therapeutic agents and diagnostic agents and a listing of species within each class can be found in Martindale, The Extra Pharmacopoeia, Twenty-ninth Edition, The Pharmaceutical Press, London, 1989 which is incorporated herein by reference and made a part hereof. The therapeutic agents and diagnostic agents are commercially available and/or can be prepared by techniques known in the art.

A cosmetic agent is any active ingredient capable of having a cosmetic activity. Examples of these active ingredients can be, inter alia, emollients, humectants, free radical-inhibiting agents, anti-inflammatories, vitamins, depigmenting agents, anti-acne agents, antiseborrhoeics, keratolytics, slimming agents, skin coloring agents and sunscreen agents, and in particular linoleic acid, retinol, retinoic acid, ascorbic acid alkyl esters, polyunsaturated fatty acids, nicotinic esters, tocopherol nicotinate, unsaponifiables of rice, soybean or shea, ceramides, hydroxy acids such as glycolic acid, selenium derivatives, antioxidants, beta-carotene, gamma-orizanol and stearyl glycerate. The cosmetics are commercially available and/or can be prepared by techniques known in the art.

Examples of nutritional supplements contemplated for use in the practice of the present invention include, but are not limited to, proteins, carbohydrates, water-soluble vitamins (e.g., vitamin C, B-complex vitamins, and the like), fat-soluble vitamins (e.g., vitamins A, D, E, K, and the like), and herbal extracts. The nutritional supplements are commercially available and/or can be prepared by techniques known in the art.

The term “pesticide” is understood to encompass herbicides, insecticides, acaricides, nematicides, ectoparasiticides and fungicides. Examples of compound classes to which the pesticide in the present invention may belong include ureas, triazines, triazoles, carbamates, phosphoric acid esters, dinitroanilines, morpholines, acylalanines, pyrethroids, benzilic acid esters, diphenylethers and polycyclic halogenated hydrocarbons. Specific examples of pesticides in each of these classes are listed in Pesticide Manual, 9th Edition, British Crop Protection Council. The pesticides are commercially available and/or can be prepared by techniques known in the art.

Preferably the organic compound or the pharmaceutically active compound is poorly water soluble. What is meant by “poorly water soluble” is a solubility of the compound in water of less than about 10 mg/mL, and preferably less than 1 mg/mL. These poorly water soluble agents are most suitable for aqueous suspension preparations since there are limited alternatives of formulating these agents in an aqueous medium.

The present invention can also be practiced with water soluble pharmaceutically active compounds, by entrapping these compounds in a solid carrier matrix (for example, polylactate-polyglycolate copolymer, albumin, starch), or by encapsulating these compounds in a surrounding vesicle that is impermeable to the pharmaceutical compound. This encapsulating vesicle can be a polymeric coating such as polyacrylate. Further, the small particles prepared from these water soluble pharmaceutical agents can be modified to improve chemical stability and control the pharmacokinetic properties of the agents by controlling the release of the agents from the particles. Examples of water soluble pharmaceutical agents include, but are not limited to, simple organic compounds, proteins, peptides, nucleotides, oligonucleotides, and carbohydrates.

The particles of the present invention have an average effective particle size of generally less than about 100 μm as measured by dynamic light scattering methods, e.g., photocorrelation spectroscopy, laser diffraction, low-angle laser light scattering (LALLS), medium-angle laser light scattering (MALLS), light obscuration methods (Coulter method, for example), rheology, or microscopy (light or electron). However, the particles can be prepared in a wide range of sizes, such as from about 20 μm to about 10 nm, from about 10 μm to about 10 mn, from about 2 μm to about 10 nm, from about 1 μm to about 10 nm, from about 400 nm to about 50 nm, from about 200 nm to about 50 nm or any range or combination of ranges therein. The preferred average effective particle size depends on factors such as the intended route of administration, formulation, solubility, toxicity and bioavailability of the compound.

To be suitable for parenteral administration, the particles preferably have an average effective particle size of less than about 7 μm, more preferably less than about 2 μm, and most preferably from about 1 μm to about 50 nm or any range or combination of ranges therein. Parenteral administration includes intravenous, intra-arterial, intrathecal, intraperitoneal, intraocular, intra-articular, intradural, intramuscular, intradermal or subcutaneous injection.

Particles sizes for oral dosage forms can be in excess of 2 μm and typically less than about 7 μm. The particles can exceed 7 μm, up to about 100 μm, provided that the particles have sufficient bioavailability and other characteristics of an oral dosage form. Oral dosage forms include tablets, capsules, caplets, soft and hard gel capsules, or other delivery vehicle for delivering a drug by oral administration.

The present invention is further suitable for providing particles of the organic compound in a form suitable for pulmonary administration. Particles sizes for pulmonary dosage forms can be in excess of 2 μm and typically less than about 10 μm. The particles in the suspension can be aerosolized and administered by a nebulizer for pulmonary administration. Alternatively, the particles can be administered as dry powder by a dry powder inhaler after removing the liquid phase from the suspension, or the dry powder can be resuspended in a non-aqueous propellant for administration by a metered dose inhaler. An example of a suitable propellant is a hydrofluorocarbon (HFC) such as HFC-134a (1,1,1,2-tetrafluoroethane) and HFC-227ea (1,1,1,2,3,3,3 -heptafluoropropane). Unlike chlorofluorcarbons (CFC's), HFC's exhibit little or no ozone depletion potential.

Dosage forms for other routes of delivery, such as nasal, topical, ophthalmic, nasal, buccal, rectal, vaginal, transdermal and the like can also be formulated from the particles made from the present invention.

Preferred microprecipitation processes for preparing the particles can be separated into three general categories. Each of the categories of processes share the steps of: (1) dissolving an organic compound in a water miscible first solvent to create a first solution, (2) mixing the first solution with a second solvent of water to precipitate the organic compound to create a pre-suspension, and (3) adding energy to the presuspension in the form of high-shear mixing or heat to provide a stable form of the organic compound having the desired size ranges defined above.

The three categories of processes are distinguished based upon the physical properties of the organic compound as determined through x-ray diffraction studies, differential scanning calorimetry (DSC) studies or other suitable study conducted prior to the energy-addition step and after the energy-addition step. In the first process category, prior to the energy-addition step the organic compound in the presuspension takes an amorphous form, a semi-crystalline form or a supercooled liquid form and has an average effective particle size. After the energy-addition step the organic compound is in a crystalline form having an average effective particle size essentially the same as that of the presuspension.

In the second process category, prior to the energy-addition step the organic compound is in a crystalline form and has an average effective particle size. After the energy-addition step the organic compound is in a crystalline form having essentially the same average effective particle size as prior to the energy-addition step but the crystals after the energy-addition step are less likely to aggregate.

The lower tendency of the organic compound to aggregate is observed by laser dynamic light scattering and light microscopy.

In the third process category, prior to the energy-addition step the organic compound is in a crystalline form that is friable and has an average effective particle size. What is meant by the term “friable” is that the particles are fragile and are more easily broken down into smaller particles. After the energy-addition step the organic compound is in a crystalline form having an average effective particle size smaller than the crystals of the pre-suspension. By taking the steps necessary to place the organic compound in a crystalline form that is friable, the subsequent energy-addition step can be carried out more quickly and efficiently when compared to an organic compound in a less friable crystalline morphology.

The energy-addition step can be carried out in any fashion wherein the pre-suspension is exposed to cavitation, shearing or impact forces. In one preferred form of the invention, the energy-addition step is an annealing step. Annealing is defined in this invention as the process of converting matter that is thermodynamically unstable into a more stable form by single or repeated application of energy (direct heat or mechanical stress), followed by thermal relaxation. This lowering of energy may be achieved by conversion of the solid form from a less ordered to a more ordered lattice structure. Alternatively, this stabilization may occur by a reordering of the surfactant molecules at the solid-liquid interface.

These three process categories will be discussed separately below. It should be understood, however, that the process conditions such as choice of surfactants or combination of surfactants, amount of surfactant used, temperature of reaction, rate of mixing of solutions, rate of precipitation and the like can be selected to allow for any drug to be processed under any one of the categories discussed next.

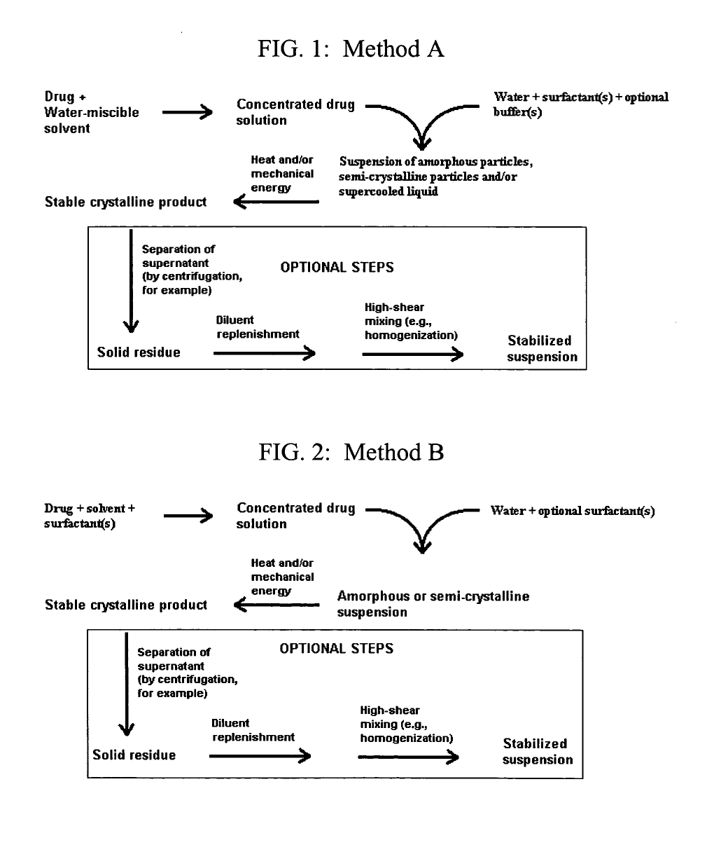

The first process category, as well as the second and third process categories, can be further divided into two subcategories, Method A, and B shown diagrammatically in FIGS. 1 and 2.

The first solvent according to the present invention is a solvent or mixture of solvents in which the organic compound of interest is relatively soluble and which is miscible with the second solvent. Examples of such solvents include, but are not limited to: polyvinylpyrrolidone, N-methyl-2-pyrrolidinone (also called N-methyl-2-pyrrolidone), 2-pyrrolidone, dimethyl sulfoxide, dimethylacetamide, lactic acid, methanol, ethanol, isopropanol, 3-pentanol, n-propanol, glycerol, butylene glycol (butanediol), ethylene glycol, propylene glycol, mono- and diacylated monoglycerides (such as glyceryl caprylate), dimethyl isosorbide, acetone, dimethylformamide, 1,4-dioxane, polyethylene glycol (for example, PEG-4, PEG-8, PEG-9, PEG-12, PEG-14, PEG-16, PEG-120, PEG-75, PEG-150, polyethylene glycol esters (examples such as PEG-4 dilaurate, PEG-20 dilaurate, PEG-6 isostearate, PEG-8 palmitostearate, PEG-150 palmitostearate), polyethylene glycol sorbitans (such as PEG-20 sorbitan isostearate), polyethylene glycol monoalkyl ethers (examples such as PEG-3 dimethyl ether, PEG-4 dimethyl ether), polypropylene glycol (PPG), polypropylene alginate, PPG-10 butanediol, PPG-10 methyl glucose ether, PPG-20 methyl glucose ether, PPG-15 stearyl ether, propylene glycol dicaprylate/dicaprate, propylene glycol laurate. A preferred first solvent is N-methyl-2-pyrrolidinone. Another preferred first solvent is lactic acid.

Method A

In Method A (see FIG. 1), the organic compound (“drug”) is first dissolved in the first solvent to create a first solution. The organic compound can be added from about 0.1% (w/v) to about 50% (w/v) depending on the solubility of the organic compound in the first solvent. Heating of the concentrate from about 30° C. to about 100° C. may be necessary to ensure total dissolution of the compound in the first solvent.

A second aqueous solvent is provided with one or more optional surface modifiers such as an anionic surfactant, a cationic surfactant, a zwitterionic surfactant, a nonionic surfactant or a biological surface active molecule added thereto. Suitable anionic surfactants include but are not limited to alkyl sulfonates, alkyl phosphates, alkyl phosphonates, potassium laurate, sodium lauryl sulfate, sodium dodecylsulfate, alkyl polyoxyethylene sulfates, sodium alginate, dioctyl sodium sulfosuccinate, phosphatidyl glycerol, phosphatidylinositol, diphosphatidylglycerol, phosphatidyl inosine, phosphatidylserine, phosphatidic acid and their salts, sodium carboxymethylcellulose, cholic acid and other bile acids (e.g., cholic acid, deoxycholic acid, glycocholic acid, taurocholic acid, glycodeoxycholic acid) and salts thereof (e.g., sodium deoxycholate, etc.).

Zwitterionic surfactants are electrically neutral but possess local positive and negative charges within the same molecule. Suitable zwitterionic surfactants include but are not limited to zwitterionic phospholipids. Suitable phospholipids include phosphatidylcholine, phosphatidylethanolamine, diacyl-glycero-phosphoethanolamine (such as dimyristoyl-glycero-phosphoethanolamine (DMPE), dipalmitoyl-glycero-phosphoethanolamine (DPPE), distearoyl-glycero-phosphoethanolamine (DSPE), and dioleolyl-glycero-phosphoethanolamine (DOPE)). Mixtures of phospholipids that include anionic and zwitterionic phospholipids may be employed in this invention. Such mixtures include but are not limited to lysophospholipids, egg or soybean phospholipid or any combination thereof. The phospholipid, whether anionic, zwitterionic or a mixture of phospholipids, may be salted or desalted, hydrogenated or partially hydrogenated or natural semisynthetic or synthetic. The phospholipid may also be conjugated with a water-soluble or hydrophilic polymer to specifically target the delivery to macrophages in the present invention. However, conjugated phospholipids may be used to target other cells or tissue in other applications. A preferred polymer is polyethylene glycol (PEG), which is also known as the monomethoxy polyethyleneglycol (mPEG). The molecule weights of the PEG can vary, for example, from 200 to 50,000. Some commonly used PEG's that are commercially available include PEG 350, PEG 550, PEG 750, PEG 1000, PEG 2000, PEG 3000, and PEG 5000. Phospholipids congugated to one or more PEGs are referred herein as a “pegylated phospholipid.” The phospholipid or the PEG-phospholipid conjugate may also incorporate a functional group which can covalently attach to a ligand including but not limited to proteins, peptides, carbohydrates, glycoproteins, antibodies, or pharmaceutically active agents. These functional groups may conjugate with the ligands through, for example, amide bond formation, disulfide or thioether formation, or biotin/streptavidin binding. Examples of the ligand-binding functional groups include but are not limited to hexanoylamine, dodecanylamine, 1,12-dodecanedicarboxylate, thioethanol, 4-(p-maleimidophenyl)butyramide (MPB), 4-(p-maleimidomethyl)cyclohexane-carboxamide (MCC), 3-(2-pyridyldithio)propionate (PDP), succinate, glutarate, dodecanoate, and biotin.

Suitable cationic surfactants include but are not limited to quaternary ammonium compounds, such as benzalkonium chloride, cetyltrimethylammonium bromide, lauryldimethylbenzylammonium chloride, acyl carnitine hydrochlorides, dimethyldioctadecylammomium bromide (DDAB), dioleyoltrimethylammonium propane (DOTAP), dimyristoyltrimethylammonium propane (DMTAP), dimethylaminoethanecarbamoyl cholesterol (DC-Chol), 1,2-diacylglycero-3-(O-alkyl)phosphocholine, O-alkylphosphatidylcholine, alkyl pyridinium halides, or long-chain alkyl amines such as, for example, n-octylamine and oleylamine.

Suitable nonionic surfactants include: glyceryl esters, polyoxyethylene fatty alcohol ethers (Macrogol and Brij), polyoxyethylene sorbitan fatty acid esters (Polysorbates), polyoxyethylene fatty acid esters (Myrj), sorbitan esters (Span), glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), polaxamines, methylcellulose, hydroxycellulose, hydroxy propylcellulose, hydroxy propylmethylcellulose, noncrystalline cellulose, polysaccharides including starch and starch derivatives such as hydroxyethylstarch (HES), polyvinyl alcohol, and polyvinylpyrrolidone. In a preferred form of the invention, the nonionic surfactant is a polyoxyethylene and polyoxypropylene copolymer and preferably a block copolymer of propylene glycol and ethylene glycol. Such polymers are sold under the tradename POLOXAMER also sometimes referred to as PLURONIC®, and sold by several suppliers including Spectrum Chemical and Ruger. Among polyoxyethylene fatty acid esters is included those having short alkyl chains. One example of such a surfactant is SOLUTOL® HS 15, polyethylene-660-hydroxystearate, manufactured by BASF Aktiengesellschaft.

Surface-active biological molecules include such molecules as albumin, casein, hirudin or other appropriate proteins. Polysaccharide biologics are also included, and consist of but are not limited to, starches, heparins, and chitosans. Other suitable surfactants include any amino acids such as leucine, alanine, valine, isoleucine, lysine, aspartic acid, glutamic acid, methionine, phenylalanine, or any derivatives of these amino acids such as, for example, amide or ester derivatives and polypeptides formed from these amino acids.

It may also be desirable to add a pH adjusting agent to the second solvent. Suitable pH adjusting agents include, but are not limited to, hydrochloric acid, sulfuric acid, phosphoric acid, monocarboxylic acids (such as, for example, acetic acid and lactic acid), dicarboxylic acids (such as, for example, succinic acid), tricarboxylic acids (such as, for example, citric acid), THAM (tris(hydroxymethyl)aminomethane), meglumine (N-methylglucosamine), sodium hydroxide, and amino acids such as glycine, arginine, lysine, alanine, histidine and leucine. The second solvent should have a pH within the range of from about 3 to about 11. The aqueous medium may additionally include an osmotic pressure adjusting agent, such as but not limited to glycerin, a monosaccharide such as dextrose, a disaccharide such as sucrose, a trisaccharide such as raffinose, and sugar alcohols such as mannitol, xylitol and sorbitol.

For oral dosage forms one or more of the following excipients may be utilized: gelatin, casein, lecithin (phosphatides), gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens™, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose phthalate, noncrystalline cellulose, magnesium aluminum silicate, triethanolamine, polyvinyl alcohol (PVA), and polyvinylpyrrolidone (PVP). Most of these excipients are described in detail in the Handbook of Pharmaceutical Excipients, published jointly by the American Pharmaceutical Association and The Pharmaceutical Society of Great Britain, the Pharmaceutical Press, 1986. The surface modifiers are commercially available and/or can be prepared by techniques known in the art. Two or more surface modifiers can be used in combination.

In a preferred form of the invention, the method for preparing small particles of an organic compound includes the steps of adding the first solution to the second solvent. The addition rate is dependent on the batch size, and precipitation kinetics for the organic compound. Typically, for a small-scale laboratory process (preparation of 1 liter), the addition rate is from about 0.05 cc per minute to about 10 cc per minute. During the addition, the solutions should be under constant agitation. It has been observed using light microscopy that amorphous particles, semi-crystalline solids, or a supercooled liquid are formed to create a pre-suspension. The method further includes the step of subjecting the pre-suspension to an annealing step to convert the amorphous particles, supercooled liquid or semicrystalline solid to a crystalline more stable solid state. The resulting particles will have an average effective particles size as measured by dynamic light scattering methods (e.g., photocorrelation spectroscopy, laser diffraction, low-angle laser light scattering (LALLS), medium-angle laser light scattering (MALLS), light obscuration methods (Coulter method, for example), rheology, or microscopy (light or electron) within the ranges set forth above).

The energy-addition step involves adding energy through sonication, homogenization, counter current flow homogenization, microfluidization, or other methods of providing impact, shear or cavitation forces. The sample may be cooled or heated during this stage. In one preferred form of the invention the annealing step is effected by a piston gap homogenizer such as the one sold by Avestin Inc. under the product designation EmulsiFlex-C160. In another preferred form of the invention, the annealing may be accomplished by ultrasonication using an ultrasonic processor such as the Vibra-Cell Ultrasonic Processor (600W), manufactured by Sonics and Materials, Inc. In yet another preferred form of the invention, the annealing may be accomplished by use of an emulsification apparatus as described in U.S. Pat. No. 5,720,551 which is incorporated herein by reference and made a part hereof.

Depending upon the rate of annealing, it may be desirable to adjust the temperature of the processed sample to within the range of from approximately −30° C. to 30° C. Alternatively, in order to effect a desired phase change in the processed solid, it may also be necessary to heat the pre-suspension to a temperature within the range of from about 30° C. to about 100° C. during the annealing step.

Method B

Method B differs from Method A in the following respects. The first difference is a surfactant or combination of surfactants is added to the first solution. The surfactants may be selected from the groups of anionic, nonionic, cationic surfactants, and surface active biological modifiers set forth above.

### COMPARATIVE EXAMPLE OF METHOD A AND METHOD B AND U.S. Pat. No. 5,780,062

U.S. Pat. No. 5,780,062 discloses a process for preparing small particles of an organic compound by first dissolving the compound in a suitable water-miscible first solvent. A second solution is prepared by dissolving a polymer and an amphiphile in aqueous solvent. The first solution is then added to the second solution to form a precipitate that consists of the organic compound and a polymer-amphiphile complex. The '062 patent does not disclose utilizing the energy-addition step of this invention in Methods A and B. Lack of stability is typically evidenced by rapid aggregation and particle growth. In some instances, amorphous particles recrystallize as large crystals. Adding energy to the pre-suspension in the manner disclosed above typically affords particles that show decreased rates of particle aggregation and growth, as well as the absence of recrystalization upon product storage.

Methods A and B are further distinguished from the process of the '062 patent by the absence of a step of forming a polymer-amphiphile complex prior to precipitation. In Method A, such a complex cannot be formed as no polymer is added to the diluent (aqueous) phase. In Method B, the surfactant, which may also act as an amphiphile, or polymer, is dissolved with the organic compound in the first solvent. This precludes the formation of any amphiphile-polymer complexes prior to precipitation. In the '062 patent, successful precipitation of small particles relies upon the formation of an amphiphile-polymer complex prior to precipitation. The '062 patent discloses the amphiphile-polymer complex forms aggregates in the aqueous second solution. The '062 patent explains the hydrophobic organic compound interacts with the amphiphile-polymer complex, thereby reducing solubility of these aggregates and causing precipitation. In the present invention it has been demonstrated that the inclusion of the surfactant or polymer in the first solvent (Method B) leads, upon subsequent addition to second solvent, to formation of a more uniform, finer particulate than is afforded by the process outlined by the '062 patent.

To this end, two formulations were prepared and analyzed. Each of the formulations have two solutions, a concentrate and an aqueous diluent, which are mixed together and then sonicated. The concentrate in each formulation has an organic compound (itraconazole), a water miscible solvent (N-methyl-2-pyrrolidinone or NMP) and possibly a polymer (poloxamer 188). The aqueous diluent has water, a tris buffer and possibly a polymer (poloxamer 188) and/or a surfactant (sodium deoxycholate). The average particle diameter of the organic particle is measured prior to sonication and after sonication.

The first formulation A has as the concentrate itraconazole and NMP. The aqueous diluent includes water, poloxamer 188, tris buffer and sodium deoxycholate. Thus the aqueous diluent includes a polymer (poloxamer 188), and an amphiphile (sodium deoxycholate), which may form a polymer/amphiphile complex, and, therefore, is in accordance with the disclosure of the '062 patent. (However, again the '062 patent does not disclose an energy addition step.)

The second formulation B has as the concentrate itraconazole, NMP and poloxamer 188. The aqueous diluent includes water, tris buffer and sodium deoxycholate. This formulation is made in accordance with the present invention. Since the aqueous diluent does not contain a combination of a polymer (poloxamer) and an amphiphile (sodium deoxycholate), a polymer/amphiphile complex cannot form prior to the mixing step.

Table 1 shows the average particle diameters measured by laser diffraction on three replicate suspension preparations. An initial size determination was made, after which the sample was sonicated for 1 minute. The size determination was then repeated. The large size reduction upon sonication of Method A was indicative of particle aggregation.

TABLE 1

| | | | Average particle | After sonication |
|---|---|---|---|---|
| Method | Concentrate | Aqueous Diluent | diameter (microns) | (1 minute) |
| A | itraconazole (18%), N- | poloxamer 188 | 18.7 | 2.36 |
| | methyl-2-pyrrolidinone (6 mL) | (2.3%), sodium deoxycholate | 10.7 | 2.46 |
| | | (0.3%) tris buffer (5 mM, | 12.1 | 1.93 |
| | | pH 8)water (qs to 94 mL) | | |
| B | itraconazole (18%)poloxamer | sodium deoxycholate | 0.194 | 0.198 |
| | 188 (37%)N-methyl-2- | (0.3%) tris buffer (5 mM, | 0.178 | 0.179 |
| | pyrrolidinone (6 mL) | pH 8)water (qs to 94 mL) | 0.181 | 0.177 |

A drug suspension resulting from application of the processes described in this invention may be administered directly as an injectable solution, provided Water for Injection is used in formulation and an appropriate means for solution sterilization is applied. Sterilization may be accomplished by separate sterilization of the drug concentrate (drug, solvent, and optional surfactant) and the diluent medium (water, and optional buffers and surfactants) prior to mixing to form the pre-suspension with subsequent steps conducted under aseptic conditions. Sterilization may also be accomplished by methods well known in the art such as steam or heat sterilization, gamma irradiation and the like. Another method for sterilization is high pressure sterilization. Other sterilization methods, especially for particles in which greater than 99% of the particles are less than 200 nm, would also include pre-filtration first through a 3.0 micron filter followed by filtration through a 0.45-micron particle filter, followed by steam or heat sterilization or sterile filtration through two redundant 0.2-micron membrane filters.

Optionally, a solvent-free suspension may be produced by solvent removal after precipitation. This can be accomplished by centrifugation, dialysis, diafiltration, force-field fractionation, high-pressure filtration or other separation techniques well known in the art. Complete removal of N-methyl-2-pyrrolidinone was typically carried out by one to three successive centrifugation runs; after each centrifugation (18,000 rpm for 30 minutes) the supernatant was decanted and discarded. A fresh volume of the suspension vehicle without the organic solvent was added to the remaining solids and the mixture was dispersed by homogenization. It will be recognized by others skilled in the art that other high-shear mixing techniques could be applied in this reconstitution step. Alternatively, the solvent-free particles can be formulated into various dosage forms as desired for a variety of administrative routes, such as oral, pulmonary, nasal, topical, intramuscular, and the like.

Furthermore, any undesired excipients such as surfactants may be replaced by a more desirable excipient by use of the separation methods described in the above paragraph. The solvent and first excipient may be discarded with the supernatant after centrifugation or filtration. A fresh volume of the suspension vehicle without the solvent and without the first excipient may then be added. Alternatively, a new surfactant may be added. For example, a suspension consisting of drug, N-methyl-2-pyrrolidinone (solvent), poloxamer 188 (first excipient), sodium deoxycholate, glycerol and water may be replaced with phospholipids (new surfactant), glycerol and water after centrifugation and removal of the supernatant.

I. First Process Category

The methods of the first process category generally include the step of dissolving the organic compound in a water miscible first solvent followed by the step of mixing this solution with an aqueous solvent to form a presuspension wherein the organic compound is in an amorphous form, a semicrystalline form or in a supercooled liquid form as determined by x-ray diffraction studies, DSC, light microscopy or other analytical techniques and has an average effective particle size within one of the effective particle size ranges set forth above. The mixing step is followed by an energy-addition step and, in a preferred form of the invention an annealing step.

II. Second Process Category

The methods of the second processes category include essentially the same steps as in the steps of the first processes category but differ in the following respect. An x-ray diffraction, DSC or other suitable analytical techniques of the presuspension shows the organic compound in a crystalline form and having an average effective particle size. The organic compound after the energy-addition step has essentially the same average effective particle size as prior to the energy-addition step but has less of a tendency to aggregate into larger particles when compared to that of the particles of the presuspension. Without being bound to a theory, it is believed the differences in the particle stability may be due to a reordering of the surfactant molecules at the solid-liquid interface.

III. Third Process Category

The methods of the third category modify the first two steps of those of the first and second processes categories to ensure the organic compound in the presuspension is in a friable form having an average effective particle size (e.g., such as slender needles and thin plates). Friable particles can be formed by selecting suitable solvents, surfactants or combination of surfactants, the temperature of the individual solutions, the rate of mixing and rate of precipitation and the like. Friability may also be enhanced by the introduction of lattice defects (e.g., cleavage planes) during the steps of mixing the first solution with the aqueous solvent. This would arise by rapid crystallization such as that afforded in the precipitation step. In the energy-addition step these friable crystals are converted to crystals that are kinetically stabilized and having an average effective particle size smaller than those of the presuspension. Kinetically stabilized means particles have a reduced tendency to aggregate when compared to particles that are not kinetically stabilized. In such instance the energy-addition step results in a breaking up of the friable particles. By ensuring the particles of the presuspension are in a friable state, the organic compound can more easily and more quickly be prepared into a particle within the desired size ranges when compared to processing an organic compound where the steps have not been taken to render it in a friable form.

In addition to the microprecipitation methods described above, any other known precipitation methods for preparing submicron sized particles or nanoparticles in the art can be used in conjunction with the present invention. The following is a description of examples of other precipitation methods. The examples are for illustration purposes, and are not intended to limit the scope of the present invention.

Emulsion Precipitation Methods

One suitable emulsion precipitation technique is disclosed in the co-pending and commonly assigned U.S. Ser. No. 09/964,273, which is incorporated herein by reference and is made a part hereof. In this approach, the process includes the steps of: (1) providing a multiphase system having an organic phase and an aqueous phase, the organic phase having a pharmaceutically effective compound therein; and (2) sonicating the system to evaporate a portion of the organic phase to cause precipitation of the compound in the aqueous phase and having an average effective particle size of less than about 2 μm. The step of providing a multiphase system includes the steps of: (1) mixing a water immiscible solvent with the pharmaceutically effective compound to define an organic solution, (2) preparing an aqueous based solution with one or more surface active compounds, and (3) mixing the organic solution with the aqueous solution to form the multiphase system. The step of mixing the organic phase and the aqueous phase can include the use of piston gap homogenizers, colloidal mills, high speed stirring equipment, extrusion equipment, manual agitation or shaking equipment, microfluidizer, or other equipment or techniques for providing high shear conditions. The crude emulsion will have oil droplets in the water of a size of approximately less than 1 μm in diameter. The crude emulsion is sonicated to define a microemulsion and eventually to define a submicron sized particle suspension.

Another approach to preparing submicron sized particles is disclosed in co-pending and commonly assigned U.S. Ser. No. 10/183,035, which is incorporated herein by reference and made a part hereof. The process includes the steps of: (1) providing a crude dispersion of a multiphase system having an organic phase and an aqueous phase, the organic phase having a pharmaceutical compound therein; (2) providing energy to the crude dispersion to form a fine dispersion; (3) freezing the fine dispersion; and (4) lyophilizing the fine dispersion to obtain submicron sized particles of the pharmaceutical compound. The step of providing a multiphase system includes the steps of: (1) mixing a water immiscible solvent with the pharmaceutically effective compound to define an organic solution; (2) preparing an aqueous based solution with one or more surface active compounds; and (3) mixing the organic solution with the aqueous solution to form the multiphase system. The step of mixing the organic phase and the aqueous phase includes the use of piston gap homogenizers, colloidal mills, high speed stirring equipment, extrusion equipment, manual agitation or shaking equipment, microfluidizer, or other equipment or techniques for providing high shear conditions.

Solvent Anti-Solvent Precipitation

Suitable solvent anti-solvent precipitation techniques are disclosed in U.S. Pat. Nos. 5,118,528 and 5,100,591 which are incorporated herein by reference and made a part hereof. The process includes the steps of: (1) preparing a liquid phase of a biologically active substance in a solvent or a mixture of solvents to which may be added one or more surfactants; (2) preparing a second liquid phase of a non-solvent or a mixture of non-solvents, the non-solvent is miscible with the solvent or mixture of solvents for the substance; (3) adding together the solutions of (1) and (2) with stirring; and (4) removing of unwanted solvents to produce a colloidal suspension of nanoparticles. The '528 patent discloses that it produces particles of the substance smaller than 500 nm without the supply of energy.

Phase Inversion Precipitation

One suitable phase inversion precipitation is disclosed in U.S. Pat. Nos. 6,235,224, 6,143,211 and U.S. patent application No. 2001/0042932 which are incorporated herein by reference and made a part hereof. Phase inversion is a term used to describe the physical phenomena by which a polymer dissolved in a continuous phase solvent system inverts into a solid macromolecular network in which the polymer is the continuous phase. One method to induce phase inversion is by the addition of a nonsolvent to the continuous phase. The polymer undergoes a transition from a single phase to an unstable two phase mixture: polymer rich and polymer poor fractions. Micellar droplets of nonsolvent in the polymer rich phase serve as nucleation sites and become coated with polymer. The '224 patent discloses that phase inversion of polymer solutions under certain conditions can bring about spontaneous formation of discrete microparticles, including nanoparticles. The '224 patent discloses dissolving or dispersing a polymer in a solvent. A pharmaceutical agent is also dissolved or dispersed in the solvent. For the crystal seeding step to be effective in this process it is desirable the agent is dissolved in the solvent. The polymer, the agent and the solvent together form a mixture having a continuous phase, wherein the solvent is the continuous phase. The mixture is then introduced into at least tenfold excess of a miscible nonsolvent to cause the spontaneous formation of the microencapsulated microparticles of the agent having an average particle size of between 10 nm and 10 μm. The particle size is influenced by the solvent:nonsolvent volume ratio, polymer concentration, the viscosity of the polymer-solvent solution, the molecular weight of the polymer, and the characteristics of the solvent-nonsolvent pair. The process eliminates the step of creating microdroplets, such as by forming an emulsion, of the solvent. The process also avoids the agitation and/or shear forces.

pH Shift Precipitation

pH shift precipitation techniques typically include a step of dissolving a drug in a solution having a pH where the drug is soluble, followed by the step of changing the pH to a point where the drug is no longer soluble. The pH can be acidic or basic, depending on the particular pharmaceutical compound. The solution is then neutralized to form a presuspension of submicron sized particles of the pharmaceutcially active compound. One suitable pH shifting precipitation process is disclosed in U.S. Pat. No. 5,665,331, which is incorporated herein by reference and made a part hereof. The process includes the step of dissolving of the pharmaceutical agent together with a crystal growth modifier (CGM) in an alkaline solution and then neutralizing the solution with an acid in the presence of suitable surface-modifying surface-active agent or agents to form a fine particle dispersion of the pharmaceutical agent. The precipitation step can be followed by steps of diafiltration clean-up of the dispersion and then adjusting the concentration of the dispersion to a desired level. This process reportedly leads to microcrystalline particles of Z-average diameters smaller than 400 nm as measured by photon correlation spectroscopy.

Other examples of pH shifting precipitation methods are disclosed in U.S. Pat. Nos. 5,716,642; 5,662,883; 5,560,932; and 4,608,278, which are incorporated herein by reference and are made a part hereof.

Infusion Precipitation Method

Suitable infusion precipitation techniques are disclosed in the U.S. Pat. Nos. 4,997,454 and 4,826,689, which are incorporated herein by reference and made a part hereof. First, a suitable solid compound is dissolved in a suitable organic solvent to form a solvent mixture. Then, a precipitating nonsolvent miscible with the organic solvent is infused into the solvent mixture at a temperature between about −10° C. and about 100° C. and at an infusion rate of from about 0.01 ml per minute to about 1000 ml per minute per volume of 50 ml to produce a suspension of precipitated non-aggregated solid particles of the compound with a substantially uniform mean diameter of less than 10 μm. Agitation (e.g., by stirring) of the solution being infused with the precipitating nonsolvent is preferred. The nonsolvent may contain a surfactant to stabilize the particles against aggregation. The particles are then separated from the solvent. Depending on the solid compound and the desired particle size, the parameters of temperature, ratio of nonsolvent to solvent, infusion rate, stir rate, and volume can be varied according to the invention. The particle size is proportional to the ratio of nonsolvent:solvent volumes and the temperature of infusion and is inversely proportional to the infusion rate and the stirring rate. The precipitating nonsolvent may be aqueous or non- aqueous, depending upon the relative solubility of the compound and the desired suspending vehicle.

Temperature Shift Precipitation

Temperature shift precipitation technique, also known as the hot-melt technique, is disclosed in U.S. Pat. No. 5,188,837 to Domb, which is incorporated herein by reference and made a part hereof. In an embodiment of the invention, lipospheres are prepared by the steps of: (1) melting or dissolving a substance such as a drug to be delivered in a molten vehicle to form a liquid of the substance to be delivered; (2) adding a phospholipid along with an aqueous medium to the melted substance or vehicle at a temperature higher than the melting temperature of the substance or vehicle; (3) mixing the suspension at a temperature above the melting temperature of the vehicle until a homogenous fine preparation is obtained; and then (4) rapidly cooling the preparation to room temperature or below.

Solvent Evaporation Precipitation

Solvent evaporation precipitation techniques are disclosed in U.S. Pat. No. 4,973,465 which is incorporated herein by reference and made a part hereof. The '465 patent discloses methods for preparing microcrystals including the steps of: (1) providing a solution of a pharmaceutical composition and a phospholipid dissolved in a common organic solvent or combination of solvents, (2) evaporating the solvent or solvents and (3) suspending the film obtained by evaporation of the solvent or solvents in an aqueous solution by vigorous stirring. The solvent can be removed by adding energy to the solution to evaporate a sufficient quantity of the solvent to cause precipitation of the compound. The solvent can also be removed by other well known techniques such as applying a vacuum to the solution or blowing nitrogen over the solution.

Reaction Precipitation

Reaction precipitation includes the steps of dissolving the pharmaceutical compound into a suitable solvent to form a solution. The compound should be added in an amount at or below the saturation point of the compound in the solvent. The compound is modified by reacting with a chemical agent or by modification in response to adding energy such as heat or UV light or the like to such that the modified compound has a lower solubility in the solvent and precipitates from the solution.

Compressed Fluid Precipitation

A suitable technique for precipitating by compressed fluid is disclosed in WO 97/14407 to Johnston, which is incorporated herein by reference and made a part hereof. The method includes the steps of dissolving a water-insoluble drug in a solvent to form a solution. The solution is then sprayed into a compressed fluid, which can be a gas, liquid or supercritical fluid. The addition of the compressed fluid to a solution of a solute in a solvent causes the solute to attain or approach supersaturated state and to precipitate out as fine particles. In this case, the compressed fluid acts as an anti-solvent which lowers the cohesive energy density of the solvent in which the drug is dissolved.

Alternatively, the drug can be dissolved in the compressed fluid which is then sprayed into an aqueous phase. The rapid expansion of the compressed fluid reduces the solvent power of the fluid, which in turn causes the solute to precipitate out as fine particles in the aqueous phase. In this case, the compressed fluid acts as a solvent.

Other Methods for Preparing Particles

The particles of the present invention can also be prepared by mechanical grinding of the active agent. Mechanical grinding include such techniques as jet milling, pearl milling, ball milling, hammer milling, fluid energy milling or wet grinding techniques such as those disclosed in U.S. Pat. No. 5,145,684, which is incorporated herein by reference and made a part hereof.

Another method to prepare the particles of the present invention is by suspending an active agent. In this method, particles of the active agent are dispersed in an aqueous medium by adding the particles directly into the aqueous medium to derive a pre-suspension. The particles are normally coated with a surface modifier to inhibit the aggregation of the particles. One or more other excipients can be added either to the active agent or to the aqueous medium.

Polymorph Control

The present invention further provides additional steps for controlling the crystal structure of the pharmaceutically-active compound to ultimately produce a suspension of the compound in the desired size range and a desired crystal structure. What is meant by the term “crystal structure” is the arrangement of the atoms within the unit cell of the crystal. Pharmaceutically-active compounds that can be crystallized into different crystal structures are said to be polymorphic. Identification of polymorphs is important step in drug formulation since different polymorphs of the same drug can show differences in solubility, therapeutic activity, bioavailabilty, and suspension stability. Accordingly, it is important to control the polymorphic form of the compound for ensuring product purity and batch-to-batch reproducibility.

The steps to control the polymorphic form of the compound includes seeding the first solution, the second solvent or the pre-suspension to ensure the formation of the desired polymorph. Seeding includes using a seed compound or adding energy. In a preferred form of the invention, the seed compound is the pharmaceutically-active compound in the desired polymorphic form. Alternatively, the seed compound can also be an inert impurity or an organic compound with a structure similar to that of the desired polymorph such as a bile salt.

The seed compound can be precipitated from the first solution. This method includes the steps of adding the pharmaceutically-active compound in sufficient quantity to exceed the solubility of the pharmaceutically-active compound in the first solvent to create a supersaturated solution. The supersaturated solution is treated to precipitate the pharmaceutically-active compound in the desired polymorphic form. Treating the supersaturated solution includes aging the solution for a time period until the formation of a crystal or crystals is observed to create a seeding mixture. It is also possible to add energy to the supersaturated solution to cause the pharmaceutically-active compound to precipitate out of the solution in the desired polymorph. The energy can be added in a variety of ways including the energy addition steps described above. Further energy can be added by heating or exposing the pre-suspension to electromagnetic energy, particle beam or electron beam sources. The electromagnetic energy includes using a laser beam, dynamic electromagnetic energy, or other radiation sources. It is further contemplated utilizing ultrasound, static electric field and a static magnetic field as the energy addition source.

In a preferred form of the invention, the method for producing seed crystals from an aged supersaturated solution includes the steps of: (i) adding a quantity of the pharmaceutically-active compound to the first organic solvent to create a supersaturated solution, (ii) aging the supersaturated solution to form detectable crystals to create a seeding mixture; and (iii) mixing the seeding mixture with the second solvent to precipitate the pharmaceutically-active compound to create a pre-suspension. The pre-suspension can then be further processed as described in detail above to provide an aqueous suspension of the pharmaceutically-active compound in the desired polymorph and in the desired size range.

Seeding can also be accomplished by adding energy to the first solution, the second solvent or the pre-suspension provided that the exposed liquid or liquids contain the pharmaceutically active compound or a seed material. The energy can be added in the same fashion as described above for the supersaturated solution.

Accordingly, the present invention provides a composition of matter of a pharmaceutically active compound in a desired polymorphic form essentially free of the unspecified polymorph or polymorphs. It is contemplated the methods of this invention can apply used to selectively produce a desired polymorph for numerous pharmaceutically active compounds.

Small-particle Pharmaceutical Formulations for Anticonvulsant (antiseizure) and Antidementia and Immunosuppresant Therapy

Seizures are caused by chemical imbalances in neuronal activation and inhibition, resulting in excess electrical discharge. The result is an electrical cascade that interferes with normal function. The standard treatment for seizure control is to administer drugs that regulate these neurochemical processes. Major anticonvulsant classes, in this regard, are the tricyclic class (carbamazepine, oxcarbazepine, etc.), gamma-aminobutyric acid analogs (e.g., vigabatrin and gabapentin), benzodiazepines (e.g., diazepam, clonazepam), hydantoins (e.g., diphenylhydantoin), barbiturates (e.g., phenobarbital), phenyltriazines (e.g., lamotrigine) and newer drugs such as topiramate and levetiracetam. Approximately 70-80% of epilepsy sufferers can completely control seizures with a single drug. Others may require a combination of two or more drugs. Unfortunately, approximately 20% of patients still have seizures that are resistant to all currently available drugs. It is thought that by enabling higher drug loading in some cases, many of these resistant seizures may be controlled. Specific anticonvulsant agents include: carbamazepine (Tegretol(R)), oxcarbazepine (Trileptal(R)), topiramate, vigabatrin, tiagabine, progabide, baclofen, 10,11 -dihydro- 10-hydroxycarbamazepine (MHD), lamotrigine (Lamictal(R)), phenytoin (Dilantin(R)), Phenobarbital, primidone, diazepam, clonazepam, lorezapam, clorazepate and felbamate. As with many CNS (central nervous system) drugs, activity of many antiseizure medications is related to their ability to penetrate the blood-brain barrier (BBB), thus requiring some degree of hydrophobicity. This translates into low aqueous solubility for a number of these medications. Examples include the benzodiazepines, tricyclics, hydantoins and barbiturates. Carbamazepine has received much attention for its ability to not only treat epilepsy but potentially other CNS disorders such as dementia.

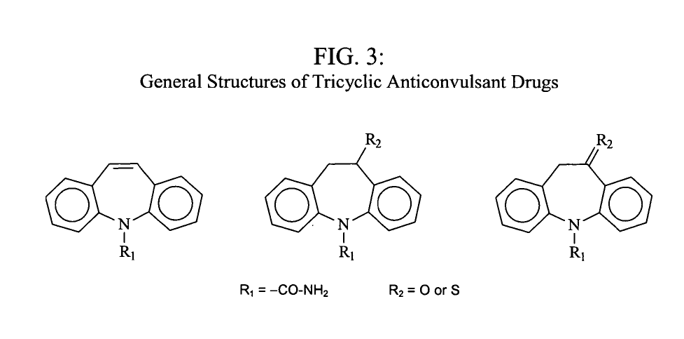

Anticonvulsants can be formulated as small-particle suspensions for pharmaceutical use. The advantages of these formulations include potentially higher drug loading with the possibility of minimizing side effects such as drowsiness, fatigue, dizziness, nystagmus or nausea. A preferred embodiment of this invention entails formulations of tricyclic anticonvulvants having the general structure shown in FIG. 3.

Antidementia agents include tranquillizers, antidepressants and anxiety-relieving agents. Specific tranquillizers include: Chlorpromazine (Largactil), Clopenthixol (Clopixol), Fluphenazine (Modecate), Haloperidol (Haldol, Serance), Olanzapine (Zyprexa), Promazine (Sparine), Quetiapine (Seroquel), Risperidone (Risperdal), Sulpiride (Dolmatil, Sulparex, Sulpatil), Thioridazine (Melleril) and Trifluoroperazine (Stelazine). Specific antidepressants include: Amitryptiline (Lentizol, Tryptizol), Amoxapine (Asendis), Citalopram (Cipramil), Dothiepin (Prothiaden), Doxepin (Sinequan), Fluoxetine (Prozac), Fluvoxamine (Faverin), Imipramine (Tofranil), Lofepramine (Gamanil), Mirtazipine (Zispin), Nefazodone (Dutonin), Nortyrptiline (Allegron), Paroxetine (Seroxat), Reboxetine (Edronax), Sertraline (Lustral) and Venlafaxine (Effexor). Specific anxiety-relieving drugs, Alprazolam (Xanax), Chlordiazepoxide (Librium), Diazepam (Valium), Lorazepam (Ativan) and Oxazepam (Oxazepam). Specific hypnotics include: Chlormethiazole (Heminevrin), Flurazepam (Dalmane), Nitrazepam (Mogadon), Temazepam (Normison), Zopiclone (Zimovane) and Zolpidem (Stilnoct).

Specific immunosuppessants include cyclosporin and its derivatives and metabolites including, but not limited to, cyclosporin A, mycophenolate mofetil (CellCept(R)), tacrolimus (Prograf(R)), sirolimus (Rapamune(R)), corticosteroids (e.g., prednisolone, methylprednisolone, cortisone, fluticasone, beclomethasone, hydrocortisone), azathioprine (Imuran(R)), 15-deoxyspergualin and leflunomide.

### EXAMPLES

### Example 1

### Preparation of 1% Carbamazepine Suspension with Phospholipid Surface Coating (from U.S. patent application US2003/031719A1)

2.08 g of carbamazepine was dissolved into 10 mL of N-methyl-2-pyrrolidinone (NMP). 1.0 mL of this concentrate was subsequently dripped at 0.1 mL/min into 20 mL of a stirred solution of 1.2% lecithin and 2.2% glycerin. As used in this patent application “percent” or “%” refers to percent weight/volume. The temperature of the lecithin system was held at 2-5° C. during the entire addition. The predispersion was next homogenized cold (5-15° C.) for 35 minutes at 15,000 psi. The pressure was increased to 23,000 psi and the homogenization was continued for another 20 minutes. The particles produced by the process had a mean diameter of 0.881 microns with 99% of the particles being less than 2.4 microns.

### Example 2

### Preparation of 1% Carbamazepine Suspension With Solutol® (Polyethyleneglycol-660, 12-hydroxystearate) (from U.S. patent application US2003/031719A1)

A drug concentrate of 20% carbamazepine and 5% glycodeoxycholic acid in N-methyl-2-pyrrolidinone was prepared. The microprecipitation step involved adding the drug concentrate to the receiving solution (distilled water) at a rate of 0.1 mL/min. The receiving solution was stirred at 500 rpm and maintained at approximately 4° C. during precipitation. After precipitation, the final ingredient concentrations were 1% carbamazepine and 0.25% glycodeoxycholate. The drug crystals were examined under a light microscope using positive phase contrast (at 400×magnification). The precipitate consisted of fine needles approximately 2.5 microns in diameter and ranging from 50-150 microns in length. Comparison of the precipitate with the raw material before precipitation reveals that the precipitation step in the presence of surface modifier (glycodeoxycholic acid) results in very slender crystals that are much thinner than the starting raw material. Homogenization of the precipitate (Avestin C-5 piston-gap homogenizer) at approximately 20,000 psi for approximately 15 minutes resulted in small particles, less than 1 micron in size and largely unaggregated.

The above process was scaled up to make a 2L suspension. After the precipitation step, the precipitate was homogenized (Avestin C-160 piston-gap homogenizer) at approximately 25,000 psi for approximately 20 passes. An aliquot of this nanosuspension was centrifuged and the supernatant replaced with a solution consisting of 0.125% Solutole® (polyethylene glycol 660, 12-hydroxystearate ester). After centrifugation and supernatant replacement, the suspension ingredient concentrations were 1% carbamazepine and 0.125% Solutol®. The samples were re-homogenized by a piston-gap homogenizer and stored at 5° C. After 3 months storage, the suspension had a mean particle size of 0.80 microns with 99% of the particles less than 1.98 microns. Numbers reported are an average of two Horiba (laser diffraction) measurements performed without sonication.

A representative batch of the above formulation was tested for particle size by laser diffraction at the end of 6 months of storage (5 and 25° C.) and revealed particle sizes that were still within the desired size range of 200 nm to 5 microns. Mean (5° C.)=0.926 micron; Mean (25° C.)=0.938 micron. Cumulative 99% diameter (5° C) =2.72 microns; Cumulative 99% diameter (25° C.)=2.71 microns.

### Example 3

### Preparation of 1% Carbamazepine Suspension with a Bile Salt and Polyether Surfactant

A drug concentrate comprising 20% carbamazepine and 5% glycodeoxycholic acid in N-methyl-2-pyrrolidinone was prepared. The microprecipitation step involved adding the drug concentrate to the receiving solution (distilled water) at a rate of 10 mL/min. The receiving solution was stirred and maintained at approximately 5° C. during precipitation. After precipitation, the final ingredient concentrations were 1% carbamazepine and 0.25% glycodeoxycholate. The precipitate was then homogenized (Avestin C-160 piston-gap homogenizer) at approximately 25,000 psi for approximately 20 passes. An aliquot of this nanosuspension was centrifuged and the supernatant replaced with a solution consisting of 0.06% glycodeoxycholate and 0.06% Poloxamer 188. After centrifugation and supernatant replacement, the suspension ingredient concentrations were 1% carbamazepine, 0.06% glycodeoxycholate, and 0.06% Poloxamer 188. The suspension was re-homogenized using a piston-gap homogenizer and stored at 5° C. After 3 months storage, the suspension had a mean particle size of 0.52 microns with 99% of the particles less than 1.15 microns. Numbers reported are an average of two Horiba (laser diffraction) measurements performed without sonication.

### Example 4

### Preparation of 1% Carbamazepine Suspension with a Phospholipid Surfactant Combination

Ingredients:

1% Carbamazepine

1.5% Lipoid E80

0.4% mPEG-DSPE (MW=2000)

0.14% sodium phosphate dibasic

2.25% glycerin

Distilled water (80 mL), 2.26 g of glycerin, 1.50 g of Lipoid E80, 0.40 g of mPEG-DSPE, and 0.14 g of sodium phosphate dibasic, were combined in a beaker and mixed with a high shear mixer until all the solids were dissolved. 1 g of carbamazepine powder was added to the surfactant solution and mixed with a high shear mixer until all of the drug powder was wetted and dispersed. The pH of the suspension was adjusted to 8.7 and diluted to a volume of 100 mL with distilled water. The suspension was homogenized at a pressure of 25,000 psi for 94 minutes, or 30 homogenization cycles. The suspension was maintained at approximately 10° C. for the entire homogenization. The final pH of the suspension was 8.3 pH units. The suspension was filled into 2 mL glass vials, flushed with nitrogen gas, and sealed with rubber stoppers. Samples were stored at 5° C. and at 25° C.

Particle Size Stability: Three samples were tested at each interval and temperature for particle size distribution by laser light scattering. The results listed below are the means of the three samples.

TABLE 2
Particle Size of Formulation from Example
4 versus Storage at 5 and 25° C.

| | 5° C. | | 25° C. | |
|---|---|---|---|---|
| Sample | Mean | 99 percentile | Mean | 99 percentile |
| Initial | 0.997 μm | 2.492 μm | 0.997 μm | 2.492 μm |
| 1 month | 1.027 | 2.718 | 1.015 | 2.828 |
| 2 months | 1.026 | 2.776 | 1.185 | 2.998 |
| 3 months | 1.001 | 2.684 | 1.035 | 2.807 |

Chemical Stability: Two samples were analyzed at each interval and temperature for the concentration of carbamazepine by high performance liquid chromatography. No significant change was observed in the drug concentrations over time.

Dissolution: Samples of the homogenized suspension were shown to completely dissolve in less than 30 seconds in Sorensen's buffer at 37° C., to give a dissolved drug concentration of about 111 ppm.

### Example 5

### Preparation of 1% Carbamazepine Suspension with Albumin

Ingredients:

1% Carbamazepine

5% Albumin (Human)

1 g of carbamazepine powder was added to 80 mL of a 5% albumin solution and mixed with a high shear mixer until all of the drug powder was wetted and dispersed. The mixture was diluted to 100 mL with the 5% albumin solution. The suspension was homogenized at a pressure of 25,000 psi for 94 minutes, or 30 homogenization cycles. The suspension was maintained at approximately 10° C. for the entire homogenization. The suspension was filled into 2 mL glass vials, flushed with nitrogen gas, and sealed with rubber stoppers. Samples were stored frozen at −20° C.

Particle Size Stability: Three samples were tested at each interval for particle size distribution by laser light scattering. The samples were allowed to thaw completely under ambient conditions before testing. The results listed below are the means of the three samples.

TABLE 3
Particle Size Versus Storage of Formulation 5 at −20° C.

| Sample | Mean | 99 percentile |
|---|---|---|
| Initial | 0.957 μm | 2.534 μm |
| 1 month | 1.142 | 3.271 |
| 2 months | 1.104 | 2.804 |
| 3 months | 0.935 | 2.973 |

Chemical Stability: Two samples were analyzed at each interval for the concentration of carbamazepine by high performance liquid chromatography. No significant change was observed in the drug concentrations over time.

Dissolution: Samples of the homogenized suspension were shown to dissolve completely in <30 seconds in Sorensen's buffer at 37° C., to give a dissolved drug concentration of about 111 ppm.

### Example 6

### Small-particle Formulation of Cyclosporin

0.4003 g of Lipoid E80 and 1.0154 g glycerin were weighed into 100 mL ethanol and dissolved to form solution 1. 0.4032 g of Poloxamer 188 was diluted to 100 mL with water to form solution 2. 0.49906 g of cyclosporin was added to 25 mL of solution 1 to form solution 3. 10 ml of each of solution 3 and solution 2 were combined to form a mixture. 80 mL of water was rapidly added to the mixture to spontaneously precipitate small particles of cyclosporin. The suspension was homogenized using an Avestin C-5 homogenizer for about 7 minutes at about 20,000 psi. Mean particle size of the homogenized nanosuspension was about 300 nm and remained at about 300 nm after 7 days at about 5° C.

While specific embodiments have been illustrated and described, numerous modifications come to mind without departing from the spirit of the invention and the scope of protection is only limited by the scope of the accompanying claims.

1. A pharmaceutical composition of an anticonvulsant agent comprising solid particles of the agent coated with one or more surface modifiers, wherein the particles have an average effective particle size of from about 10 nm to about 100 microns.

2. The composition of claim 1, wherein the surface modifier is selected from the group consisting of: anionic surfactants, cationic surfactants, zwitterionic surfactants, nonionic surfactants, surface active biological modifiers, and combinations thereof.

3. The composition of claim 2, wherein the anionic surfactant is selected from the group consisting of: alkyl sulfonates, alkyl phosphates, alkyl phosphonates, potassium laurate, triethanolamine stearate, sodium lauryl sulfate, sodium dodecylsulfate, alkyl polyoxyethylene sulfates, sodium alginate, dioctyl sodium sulfosuccinate, sodium carboxymethylcellulose, bile acids and their salts, cholic acid, deoxycholic acid, glycocholic acid, taurocholic acid, glycodeoxycholic acid, and calcium carboxymethylcellulose.

4. The composition of claim 2, wherein the cationic surfactant is selected from the group consisting of quaternary ammonium compounds, benzalkonium chloride, cetyltrimethylammonium bromide, lauryldimethylbenzylammonium chloride, acyl carnitine hydrochlorides, dimethyldioctadecylammomium bromide, dioleyoltrimethylammonium propane, dimyristoyltrimethylammonium propane, dimethylaminoethanecarbamoyl cholesterol, 1,2-dialkylglycero-3-alkylphosphocholine, alkyl pyridinium halides, n-octylamine and oleylamine.

5. The composition of claim 2, wherein the anionic surfactant is a natural, synthetic, salted or desalted phospholipid.

6. The composition of claim 5, wherein the phospholipid is selected from the group consisting of: phosphatidylglycerol, phosphatidylinositol, phosphatidylserine, diphosphatidylglyerol, phosphatidic acid and their salts.

7. The composition of claim 2, wherein the cationic surfactant is a natural, synthetic, salted or desalted phospholipid.

8. The composition of claim 7, wherein the phospholipid is selected from the group consisting of O-alkylated phosphatidylcholines.

9. The composition of claim 2, wherein the zwitterionic surfactant is a phospholipid, and wherein the phospholipid is natural or synthetic, salted or desalted.

10. The composition of claim 9, wherein the zwitterionic phospholipid is selected from the group consisting of: dipalmitoylphosphatidylcholine, phosphatidylcholine, phosphatidylethanolamine, lysophospholipids, egg phospholipid, soybean phospholipid, diacyl-glycero-phosphoethanolamine, dimyristoyl-glycero-phosphoethanolamine, dipalmitoyl-glycero-phosphoethanolamine, distearoyl-glycero-phosphoethanolamine, and dioleolyl-glycero-phosphoethanolamine).

11. The composition of claim 1, wherein the surface modifier is a pegylated phospholipid.

12. The composition of claim 2, wherein the nonionic surfactant is selected from the group consisting of: glyceryl esters, polyoxyethylene fatty alcohol ethers, polyoxyethylene sorbitan fatty acid esters, polyoxyethylene fatty acid esters, sorbitan esters, glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers, polaxamines, methylcellulose, hydroxycellulose, hydroxy propylcellulose, hydroxy propylmethylcellulose, noncrystalline cellulose, polysaccharides, starch, starch derivatives, hydroxyethylstarch, polyvinyl alcohol, and polyvinylpyrrolidone.

13. The composition of claim 2, wherein the surface active biological modifier is selected from the group consisting of proteins, polysaccharides, and combinations thereof.

14. The composition of claim 13, wherein the polysaccharide is selected from the group consisting of starches, heparin and chitosans.

15. The composition of claim 13, wherein the protein is selected from the group consisting of albumin and casein.

16. The composition of claim 1, wherein the surface modifier comprises a bile acid or a salt thereof.

17. The composition of claim 16, wherein the bile acid or salt is selected from the group consisting of deoxycholic acid, glycocholic acid, glycodeoxycholic acid, taurocholic acid and salts of these acids.

18. The composition of claim 1, wherein the surface modifier comprises a copolymer of oxyethylene and oxypropylene.

19. The composition of claim 18, wherein the copolymer of oxyethylene and oxypropylene is a block copolymer.

20. The composition of claim 1, further comprising a pH adjusting agent.

21. The composition of claim 20, wherein the pH adjusting agent is selected from the group consisting of hydrochloric acid, sulfuric acid, phosphoric acid, acetic acid, lactic acid, succinic acid, citric acid, tris(hydroxymethyl)aminomethane, N-methylglucosamine, sodium hydroxide, glycine, arginine, lysine, alanine, histidine and leucine.

22. The composition of claim 20, wherein the pH adjusting agent is added to the composition to bring the pH of the composition within the range of from about 3 to about 11.

23. The composition of claim 1, wherein the anticonvulsant agent is a tricyclic anticonvulsant agent.

24. The composition of claim 23, wherein the tricyclic anticonvulsant agent is carbamazepine.

25. A pharmaceutical composition of an immunosuppressive agent comprising solid particles of the agent coated with one or more surface modifiers, wherein the particles have an average effective particle size of from about 10 nm to about 100 microns.

26. The composition of claim 25, wherein the surface modifier is selected from the group consisting of: anionic surfactants, cationic surfactants, zwitterionic surfactants, nonionic surfactants, surface active biological modifiers, and combinations thereof.

27. The composition of claim 26, wherein the anionic surfactant is selected from the group consisting of: alkyl sulfonates, alkyl phosphates, alkyl phosphonates, potassium laurate, triethanolamine stearate, sodium lauryl sulfate, sodium dodecylsulfate, alkyl polyoxyethylene sulfates, sodium alginate, dioctyl sodium sulfosuccinate, sodium carboxymethylcellulose, bile acids and their salts, cholic acid, deoxycholic acid, glycocholic acid, taurocholic acid, glycodeoxycholic acid, and calcium carboxymethylcellulose.

28. The composition of claim 26, wherein the cafionic surfactant is selected from the group consisting of quaternary ammonium compounds, benzalkonium chloride, cetyltrimethylammonium bromide, lauryldimethylbenzylammonium chloride, acyl carnitine hydrochlorides, dimethyldioctadecylammomium bromide, dioleyoltrimethylammonium propane, dimyristoyltrimethylammonium propane, dimethylaminoethanecarbamoyl cholesterol, 1,2-dialkylglycero-3-alkylphosphocholine, alkyl pyridinium halides, n-octylamine and oleylamine.

29. The composition of claim 26, wherein the anionic surfactant is a natural, synthetic, salted or desalted phospholipid.

30. The composition of claim 29, wherein the phospholipid is selected from the group consisting of: phosphatidylglycerol, phosphatidylinositol, phosphatidylserine, diphosphatidylglyerol, phosphatidic acid and their salts.

31. The composition of claim 26, wherein the cationic surfactant is a phospholipid, and wherein the phospholipid is natural or synthetic, salted or desalted.

32. The composition of claim 31, wherein the phospholipid is selected from the group consisting of O-alkylated phosphatidylcholines.

33. The composition of claim 26, wherein the zwitterionic surfactant is a natural, synthetic, salted or desalted phospholipid.

34. The composition of claim 33, wherein the zwitterionic phospholipid is selected from the group consisting of: dipalmitoylphosphatidylcholine, phosphatidylcholine, phosphatidylethanolamine, lysophospholipids, egg phospholipid, soybean phospholipid, diacyl-glycero-phosphoethanolamine, dimyristoyl-glycero-phosphoethanolamine, dipalmitoyl-glycero-phosphoethanolamine, distearoyl-glycero-phosphoethanolamine, and dioleolyl-glycero-phosphoethanolamine.

35. The composition of claim 25, wherein the surface modifier is a pegylated phospholipid.

36. The composition of claim 26, wherein the nonionic surfactant is selected from the group consisting of: glyceryl esters, polyoxyethylene fatty alcohol ethers, polyoxyethylene sorbitan fatty acid esters, polyoxyethylene fatty acid esters, sorbitan esters, glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers, polaxamines, methylcellulose, hydroxycellulose, hydroxy propylcellulose, hydroxy propylmethylcellulose, noncrystalline cellulose, polysaccharides, starch, starch derivatives, hydroxyethylstarch, polyvinyl alcohol, and polyvinylpyrrolidone.

37. The composition of claim 26, wherein the surface active biological modifier is selected from the group consisting of proteins, polysaccharides, and combinations thereof.

38. The composition of claim 37, wherein the polysaccharide is selected from the group consisting of starches, heparin and chitosans.

39. The composition of claim 37, wherein the protein is selected from the group consisting of albumin and casein.

40. The composition of claim 25, wherein the surface modifier comprises a bile acid or a salt thereof.

41. The composition of claim 40, wherein the bile acid or salt is selected from the group consisting of deoxycholic acid, glycocholic acid, glycodeoxycholic acid, taurocholic acid and salts of these acids.

42. The composition of claim 25, wherein the surface modifier comprises a copolymer of oxyethylene and oxypropylene.

43. The composition of claim 42, wherein the copolymer of oxyethylene and oxypropylene is a block copolymer.

44. The composition of claim 25, further comprising a pH adjusting agent.

45. The composition of claim 44, wherein the pH adjusting agent is selected from the group consisting of hydrochloric acid, sulfuric acid, phosphoric acid, acetic acid, lactic acid, succinic acid, citric acid, tris(hydroxymethyl)aminomethane, N-methylglucosamine, sodium hydroxide, glycine, arginine, lysine, alanine, histidine and leucine.

46. The composition of claim 45, wherein the pH adjusting agent is added to the composition to bring the pH of the composition within the range of from about 3 to about 11.

47. The composition of claim 25, wherein the immunosuppressive agent is selected from the group consisting of: cyclosporin, cyclosporin A, a cylcosporin derivative, a cylosporin metabolite and combinations thereof.
